(19) United States
(12) Patent Application Publication
(10) Pub. No.: US 2016/0346129 A1
(43) Pub. Date: Dec. 1, 2016

(54) Sleep Mask Having Ear Covers

(71) Applicant: Kathy Schaefer, Danville, KY (US)

(72) Inventor: Kathy Schaefer, Danville, KY (US)

(21) Appl. No.: 15/010,881

(22) Filed: Jan. 29, 2016

Related U.S. Application Data
(60) Provisional application No. 62/166,205, filed on May 26, 2015.

(51) Int. Cl. A61F 9/04; A61F 11/14

(52) U.S. Cl. CPC A61F 9/045; A61F 11/14

(57) ABSTRACT

A sleep mask having ear covers to prevent a user from being disturbed by light or noise while sleeping. The sleep mask includes a pair of ear covers that can be positioned over the user's ears to dampen sounds in order to prevent noise from disturbing the user. The ear covers are disposed on opposing ends of an ear cover strap that can be positioned over the top of a user's head. The sleep mask further includes an eye cover that covers both eyes of the user to prevent light from reaching the user's eyes. The eye cover includes an eye cover strap extending therefrom, wherein the strap can be wrapped around the back of the user's head so as to secure the eye cover on the user's head. The eye cover strap is removably securable to the ear covers of the sleep mask.

### CROSS REFERENCE TO RELATED APPLICATION

This application claims the benefit of U.S. Provisional Application No. 62/166,205 filed on May 26, 2015. The above identified patent application is herein incorporated by reference in its entirety to provide continuity of disclosure.

### BACKGROUND OF THE INVENTION

### Field of the Invention

The present invention relates to sleep masks. More specifically, the present invention provides a sleep mask having ear covers so as to prevent light and sounds from disturbing the user, particularly when the user is trying to sleep.

Many people have difficulty falling to sleep at night and remaining asleep throughout the night. Some people are more easily aroused from sleep than others, and small disturbances may cause a person to wake-up. People are commonly roused from sleep due to light from within the room, outside the room, or from the flickering of a television screen. Further, noises such as people talking in another room, listening to music, or watching television may wake the person. This is particularly problematic for people who live with one or more roommates or family members.

While conventional sleep masks may be used to help block light, such masks do not help to prevent the person from being disturbed by noises. While some people utilize ear plugs to dampen sounds, many people find inserting an ear plug into their ears to be uncomfortable. Further, ear plugs are easily lost and must be frequently replaced. Thus, a single device that helps to block both light and sounds in order to allow a user to sleep undisturbed is desired.

Devices have been disclosed in the prior art that relate to headgear for sleeping. These include devices that have been patented and published in patent application publications. These devices generally relate to headgear to be worn while sleeping for providing comfort to the user or for blocking light, such as U.S. Published Patent Application Number 2010/0186145, U.S. Published Patent Application Number 2010/0200003, U.S. Published Patent Application Number 2014/0209101, U.S. Pat. No. 5,551,087, and U.S. Pat. No. 2,696,007.

These prior art devices have several known drawbacks. The devices in the prior art relate to headgear to be worn while sleeping for providing comfort to the user. Some such devices additionally help to alleviate problems such as sleep apnea and snoring. However, these devices do not serve to block or dampen light and sounds in order to prevent the user from being disturbed by the same.

In light of the devices disclosed in the prior art, it is submitted that the present invention substantially diverges in design elements from the prior art and consequently it is clear that there is a need in the art for an improvement to existing sleep mask devices. In this regard the instant invention substantially fulfills these needs.

### SUMMARY OF THE INVENTION

In view of the foregoing disadvantages inherent in the known types of sleeping headgear now present in the prior art, the present invention provides a new sleep mask device wherein the same can be utilized for providing convenience for the user when preventing a user from being disturbed by lights or sounds while sleeping.

It is therefore an object of the present invention to provide a new and improved sleep mask comprising an eye cover for covering both eyes of the user, wherein the eye cover is disposed on an eye cover strap that can be secured around the user's head.

It is another object of the present invention to provide a sleep mask further comprising a pair of ear covers adapted to be placed over the user's ears in order to prevent the user from being disturbed by sounds.

Another object of the present invention is to provide a sleep mask comprising a strap that can be adjustably secured on the user's head via mating fasteners, such as hook and loop fastening material.

Yet another object of the present invention is to provide a sleep mask wherein the eye cover strap is removably securable to the ear covers via a fastener, such as hook and loop fastening material.

Another object of the present invention is to provide a sleep mask that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the sleep mask having ear covers.

FIG. 2 shows a perspective view of an alternate embodiment of the eye cover and strap of the sleep mask.

FIG. 3 shows a close-up view of an ear cover of the sleep mask.

FIG. 4 shows a perspective view of the sleep mask as worn by a user.

### DETAILED DESCRIPTION OF THE INVENTION

Reference is made herein to the attached drawings. Like reference numerals are used throughout the drawings to depict like or similar elements of the sleep mask. For the purposes of presenting a brief and clear description of the present invention, the preferred embodiment will be discussed as used for preventing a user from being disturbed by light or sound while sleeping. The figures are intended for representative purposes only and should not be considered to be limiting in any respect.

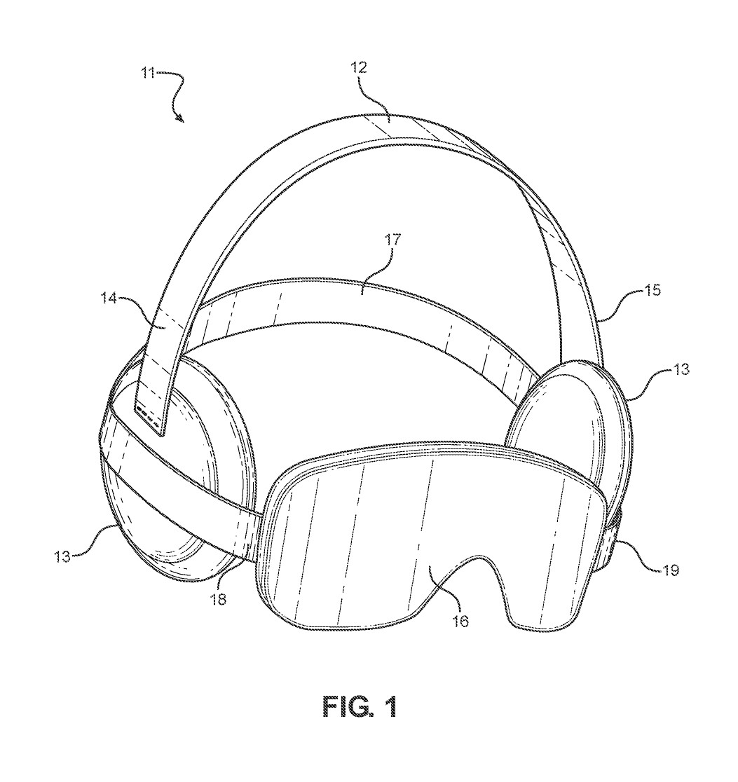

Referring now to FIG. 1, there is shown a perspective view of the sleep mask having ear covers. The sleep mask 11 comprises an eye cover 16 adapted to be positioned over the eyes of the user. The eye cover 16 is preferably shaped so as to cover both eyes of the user. In the illustrated embodiment, the eye cover 16 comprises a unitary body having a recessed lower end so that the eye cover 16 does not extend onto the user's nose. However, in alternate embodiments, the eye cover 16 may comprise a pair of covers, wherein one cover is provided for each eye and wherein the pair of covers are connected by a strap that extends between the pair of eye covers.

The eye cover 16 is preferably composed of a soft, flexible material so that the eye cover 16 conforms to the shape of the user's face. Thus, in some embodiments, the eye cover 16 is composed of a fabric material, such as cotton. In alternate embodiments, the eye cover 16 may include a hollow interior volume filled with a gel. Further, the eye cover 16 is composed of an opaque material that prevents light from passing therethrough. The eye cover 16 may further include padding to provide comfort to the user.

An eye cover strap 17 having a first end 18 and a second end 19 extends from opposing sides of the eye cover 16, wherein the eye cover strap 17 forms a loop that can be disposed around the user's head so as to secure the eye cover 12 in position over the user's eyes. In some embodiments, the eye cover strap 17 comprises an elastic material so that the eye cover strap 17 can be stretched into position around the user's head.

The sleep mask 11 further comprises a pair of ear covers 13. An ear cover strap 12 includes a first end 14 and a second end 15, wherein an ear cover 13 is positioned on each end of the ear cover strap 12. The ear cover strap 12 is preferably composed of a semi-rigid material so that it retains its shape and helps to secure the ear covers 13 in position over the user's ears. Thus, the ear cover strap 12 can be composed of a thermoplastic material. The ear cover strap 12 is adapted to extend over the top of the user's head, similar to how conventional headphones are constructed.

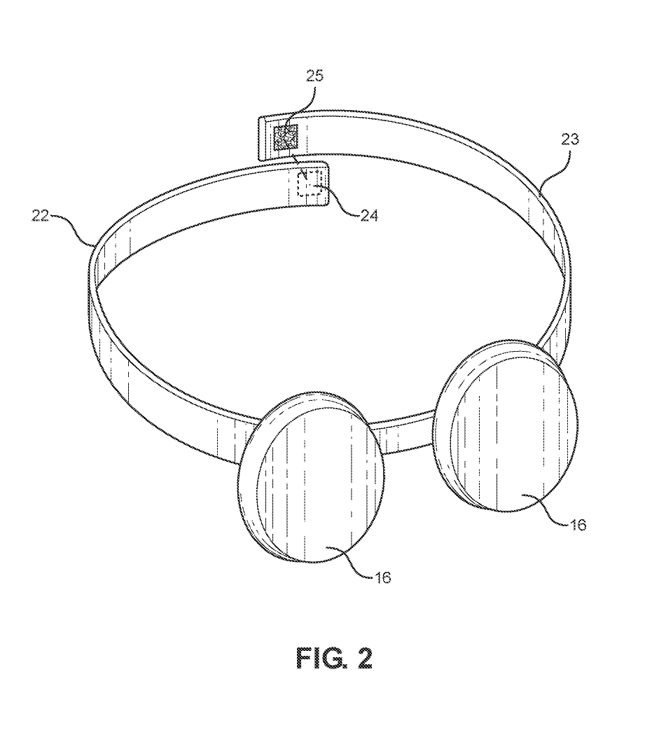

Referring now to FIG. 2, there is shown a perspective view of an alternate embodiment of the eye cover and strap of the sleep mask. In the illustrated embodiment, the eye cover 16 is divided into a pair of eye covers 16. Each eye cover 16 is shown as having a circular shape, wherein each eye cover 16 is adapted to cover one eye of the user. The pair of eye covers 16 are connected by a central strap, wherein the central strap may be a part of the eye cover strap.

In the illustrated embodiment, the eye cover strap comprises a first strap 22 and a second strap 23, wherein the straps 22, 23 extend from opposing sides of the eye cover 16. The first and second straps 22, 23 are preferably composed of a fabric material. The free ends of the first 22 and second straps 23 comprise mating fasteners 24, 25 thereon. The mating fasteners 24, 25 preferably comprise hook and loop fastening material, however, other types of fasteners may be used in alternate embodiments. In this way, the first and second strap 22, 23 can be removably secured together in an overlapping configuration at the rear of the user's head, wherein the mating fasteners 24, 25 allow the tightness of the straps to be adjusted so that the sleep mask 11 is securely positioned on the user's head.

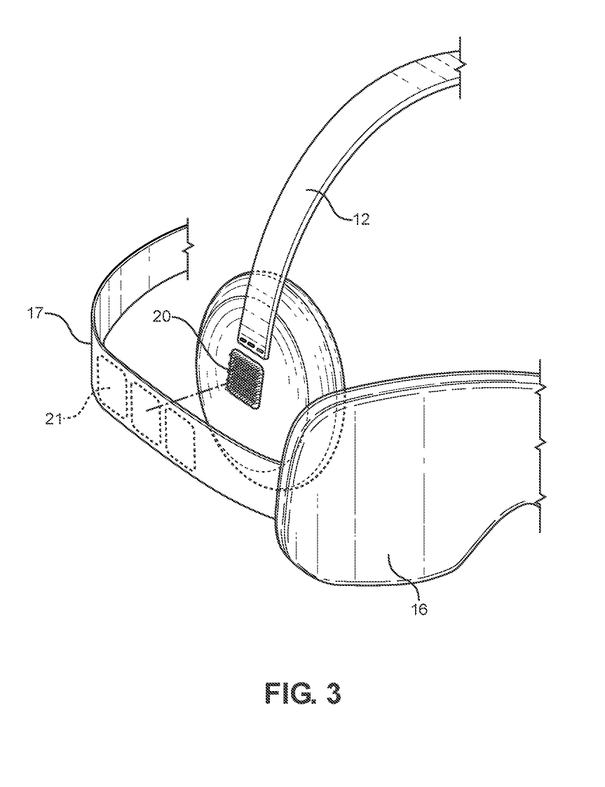

Referring now to FIG. 3, there is shown a close-up view of the ear covers of the sleep mask. The ear cover 13 preferably comprises a disk shape and is sized so as to fully cover a user's ear. Each ear cover 13 is adapted to block sound to help dampen or prevent noise from reaching the user's ears. The ear cover 13 is preferably composed of a dense material that helps to reduce or dampen sound. The ear cover 13 may be composed of one or more layers in order to dampen sound while also providing comfort to the user. It is not desired to limit the exact construction of the ear cover.

The exterior of each ear cover 13 includes a fastener 20 thereon, such as hook and loop fastening material. The fastener 20 is adapted to removably engage with a mating fastener 21 disposed on an interior surface of the eye cover strap 17. The eye cover strap 17 preferably includes a plurality of mating fasteners 21 thereon so that the user can adjustably secure the eye cover 16 on his or her head at a desired amount of tension. In alternate embodiments, other fasteners 20, 21 may be used such as snaps, claps, or buttons, among other conventional fasteners. Further, in some embodiments, the eye cover strap 17 is permanently affixed to the eye covers 16 via stitching or other permanent fastener.

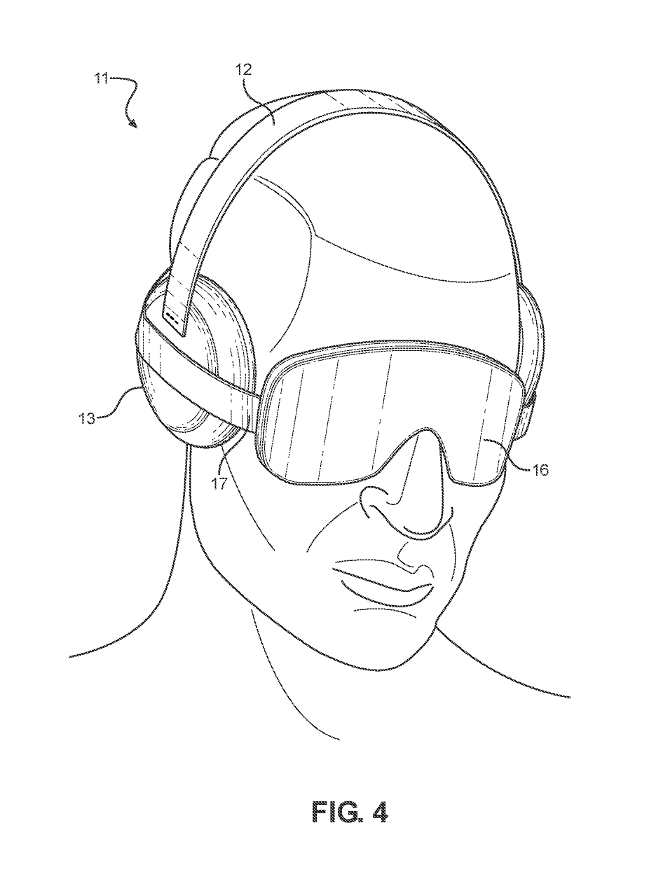

Referring now to FIG. 4, there is shown a perspective view of the sleep mask as worn by a user. In operation, the user can dispose the ear covers 13 over his or her ears with the ear cover strap 12 extending over the top of the user's head. The ear cover strap 12 provides tension so as to secure the ear covers 13 in position over the user's ears. The ear covers 13 help to dampen sounds that would otherwise disturb the user. The ear covers 13 fully cover the user's ear and are placed flush against the user's ears and the side of the user's head.

The user can position the eye cover 16 of the sleep mask 11 over his or her eyes. To secure the eye cover 16 in place, the user positions the eye cover strap 17 around the back of his or her head so as to secure the eye cover 16 in position. To provide further securement, the user secures the eye cover strap 17 to the ear covers 13 via the mating fasteners thereon. This also allows the user to adjust the positioning of the eye cover 16.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A sleep mask, comprising:
an eye cover;
an eye cover strap extending from the eye cover, wherein the eye cover strap is adapted to encircle a user's head;
an ear cover strap having a first end and a second end, wherein a first ear cover is positioned on the first end, and a second ear cover is positioned on the second end of the ear cover strap.

2) The sleep mask of claim 1, wherein:
the eye cover comprises a unitary body adapted to cover both eyes of the user.

3) The sleep mask of claim 1, wherein:
the eye cover comprises a pair of eye covers, wherein one eye cover is adapted to cover one eye of the user.

4) The sleep mask of claim 1, wherein:
the pair of ear covers each comprises a disk shape and are adapted to fully cover a user's ears.

5) The sleep mask of claim 1, wherein:
an exterior of each of the first ear cover and the second ear cover each comprises a fastener thereon;
an interior surface of the eye cover strap includes a mating fastener thereon;
wherein the fastener and the mating fastener are adapted to removably engage so as to secure the eye cover strap to the pair of ear covers.

6) The sleep mask of claim 1, wherein the ear cover strap is composed of a semi-rigid material so as to help secure the pair of ear covers in position on a user's ears.

7) The sleep mask of claim 1, wherein the eye cover strap includes a first strap and a second strap, wherein the first strap and the second strap are adapted to be removably secured together around a user's head.

8) The sleep mask of claim 1, wherein the eye cover strap is permanently affixed to the first ear cover and the second ear cover.

9) A sleep mask, comprising:
an eye cover comprising a unitary body such that it is adapted to cover both eyes of a user;
an eye cover strap extending from the eye cover, wherein the eye cover strap is adapted to encircle a user's head;
a pair of ear covers, wherein the pair of ear covers are removably securable to the eye cover strap.
